(19) United States
(12) Patent Application Publication
(10) Pub. No.: US 2007/0148249 A1
(43) Pub. Date: Jun. 28, 2007

(54) Method of preparation of oral solid dosage form with instant release of acting

(76) Inventor: Ales FRANC, Brno (CZ)

Correspondence Address: Notaro & Michalos, Suite 110, 100 Dutch Hill Road, Orangeburg, NY, 10962-2100, US

(21) Appl. No.: 10/580,185

(22) PCT Filed: Nov. 23, 2004
(86) PCT No.: PCT/CZ04/00078

§ 371 Date: May 22, 2006

(30) Foreign Application Priority Data
Nov. 25, 2003 (CZ) PV 2003-3216

(51) Int. Cl. A61K 31/473; A61K 9/14

(52) U.S. Cl. 424/489; 514/284

(57) ABSTRACT

A method of preparation of an oral solid dosage form with instant release of an active agent containing as the active agent finasteride characterized in that an aqueous suspension containing 5% to 50% by weight of finasteride, based on the total weight of the suspension, and 0.1 to 50% by weight of at least one anion surfactant, based on the weight of finasteride is milled in order to reach such distribution of particle size of finasteride that the size of 10% of particles does not exceed 2 μm, the size of 50% of particles does not exceed 7 μm, and the size of 90% of particles does not exceed 17 μm, then the obtained aqueous suspension is sprayed to a fluid bed onto a solid particle hydrophilic carrier having such distribution of particle size that the size of 90% of particles exceeds 40 μm and the size of 10 of particles exceeds 200 μm, and the size of 99% of particles does not exceed 300 μm.

### FIELD OF THE INVENTION

The present invention relates to a method used for preparation of an oral solid dosage form with instant release of the active ingredient containing finasteride as the active ingredient.

### BACKGROUND OF THE INVENTION

Finasteride is the generally accepted name for (5α,17β)-N-(1,1-dimethylethyl)-3-oxo-4-azaandrost-1-en-17-carboxamide having the structural formula 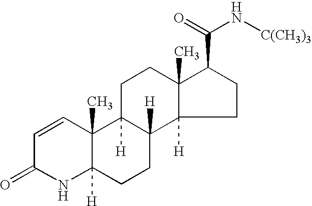
The summary formula of the substance as mentioned above is C₂₃H₃₆N₂O₂, its molecular weight is 372.55 and its melting point is 257° C. It is well soluble in chloroform and lower alcohols and almost insoluble in water. Three finasteride polymorphous forms, marked Form I, Form II, and Form M are known These polymorphous forms differ essentially only with their X-ray spectra while their important physical-chemical properties are identical. Finasteride is biologically active ingredient which affects hormonal system of the organism. For therapeutic purposes it is applied as solid oral dosage form intended for treatment of alopecia and benign prostatic hyperplasia Use of finasteride for treatment of malignant diseases is also anticipated. The mechanism of finasteride action is based on the specific inhibition of 5α-reductase, the intracellular enzyme transforming testosterone—the male sex hormone—to its effective metabolite—5α-dihydrotestosterone.

Physical-chemical parameters of finasteride, particularly its extremely low solubility in water, low wettability and high electrostatic charge of all known finasteride polymorphous forms, do not facilitate the formation of the solid dosage form with instant release of the active ingredient without involving demanding methods of pharmaceutical technology. Finasteride dissolution rate can be enhanced by enlargement of its surface and thus by reduction of the particle size. Finasteride high electrostatic charge and its non-wetting power, however, do not facilitate milling of the active ingredient even either being in the solid form or under wet conditions. The method currently used for reduction of finasteride particle size consists in controlled crystallization of finasteride obtained during te final stage of its synthesis which demands special sophisticated equipment. Manufacturing of the solid dosage form, particularly tablets, with finasteride instant release has depended so far on the use of finasteride containing very fine particles obtained using the demanding technological method mentioned above.

This invention is aimed at manufacturing of finasteride solid dosage form with instant release of the active agent enabling finasteride processing to the dosage form irrespectively of the size of its particles, i.e. processing of relatively large finasteride particles it has not been possible to use so far for the preparation of the oral dosage form with instant release of the active ingredient.

The aim as mentioned above has been reached using tie method according to this invention.

### SUMMARY OF THE INVENTION

The subject-matter of this invention is a method intended for preparation of oral solid dosage form with instant release of an active agent containing as the active agent finasteride characterized in that an aqueous suspension containing 5 to 50% by weight of finasteride, based on the total weight of the suspension, and 0.1 to 50% by weight of at least one anion surfactant, based on the weight of finasteride is milled in order to reach such distribution of particle size of finasteride that the size of 10% of particles does not exceed 2 Elm, the size of 50% of particles does not exceed 7 μm, and the size of 90% of particles does not exceed 17 μm, then the obtained aqueous suspension is sprayed to a fluid bed onto a solid particle hydrophilic carrier having such distribution of particle size that the size of 90% of particles exceeds 40 μm and the size of 10% of particles exceeds 200 μm, and the size of 99% of particles does not exceed 300 μm.

At least one substance of the following: sodium sulfosuccinate, sodium lauryl sulfate, sodium hexadecylsulfate, sodium hexadecylsulfonate, and sodium dioctylsulfosuccinate is advantageously used as anion surfactant

A hydrophilic sugar as sucrose, sorbitol, mannitol, glucose and lactose, native or modified starch, and cellulose or their mixtures, particularly a mixture of lactose, microcrystalline cellulose and modified maize starch at the weight ratio of 142:86:11 are advantageously used as the solid particle hydrophilic carrier.

The mixture obtained by the spraying of the aqueous suspension onto the solid particle hydrophilic carrier in the fluid bed is profitably mixed with 2 to 10% by weight, based on the total weight of the obtained mixture, of at least one pharmaceutically acceptable hydrophilic lubricant showing an antistatic effect, such as colloidal silicon dioxide, sodium stearyl fumarate, polyethylene glycol or sodium lauryl sulfate.

The mixture obtained by the spraying of the aqueous suspension onto the solid particle hydrophilic carrier in the fluid bed is advantageously mixed with 1 to 7% by weight, based on the total weight of the obtained mixture, of at least one pharmaceutically acceptable disintegrant, such as ultraamylopectin, cross-linked sodium carboxymethylcellulose or cross-linked polyvinylpyrrolidone.

The mixture obtained by the spraying of the aqueous suspension onto the solid particle hydrophilic carrier in the fluid bed, optionally after being mixed with at least one lubricant and/or with at least one disintegrant, is filled into capsules or sachets or is pressed into tablets.

The tablets are profitably coated with a water-soluble film or pigmented coating dispersion, particularly the dispersion of the hydrophilic coating mixture based on hydroxypropylmethylcellulose and hydroxypropylcellulose wherein the coat weight is 1 to 6% by weight based on the weight of the uncoated tablet.

The subject-matter of this invention consists in the use of a suitable tenside making finasteride wettable and thus enabling finasteride wet micronization. Said micronization within the scope of this invention represents the constituent part of preparation of the dosage form with instant release of the active ingredient. Used tenzide is dissolved in water and thus enables finasteride dispersion in aqueous medium while the obtained suspension enables its wet milling. Ultraturax mill, colloid mill, ball mil or dynomill can be obviously used for this purpose. Nevertheless, obvious wet granulation does not enable quick and sufficient finasteride release in a dissolution mediual It has been found out within the scope of the invention that the best results are obtained when said suspension is sprayed to the fluid bed onto the solid particle hydrophilic carrier with specific distribution of particle size ensuring the demanded flow properties of resulting mixture necessary for further processing of said mixture, for example, tablet-pressing. Finasteride adhesion to die surfaces occurs during tablet-pressing when obvious lubricants are used in obvious concentrations. Said effect could be avoided through the use of higher concentration of lubricants showing antistatic properties. As higher concentrations of hydrophobic lubricants, e.g. magnesium stearate, talc and stearic acid, inhibit significantly finasteride release hydrophilic lubricants with antistatic effect are used within the scope of this invention.

In the next part of the description, this invention will be explained more closely using its particular embodiment while the examples mentioned are illustrative only and does in no way limit the scope of the patent defined unambiguously by the definition of the claims and the contents of the patent description.

### BRIEF DESCRIPTION OF THE DRAWINGS

FIG. 1 is a graphical representation of finasteride release from a solid dosage form prepared using a method according to the invention and from PROSCAR generic standard.

### EXAMPLES

### Example 1

Within the scope of this invention, tablets weighing 150 mg and containing 1 mg and 5 mg of finasteride are manufactured. The composition of said tablets is set forth in Table 1 below; the contents of the individual constituents of the tablet composition are given in the weight parts.

TABLE 1

| | Tablet containing | Tablet containing |
|---|---|---|
| Constituent | 1 mg of finastride | 5 mg of finasteride |
| Finasteride | 1.00 | 5.00 |
| Aerosol OT (anion surfactant, | 0.10 | 0.50 |
| sodium sulfosuccinate) | | |
| Water | 20.00 | 45.00 |
| Starch 1500 (carrier, modified | 7.50 | 7.50 |
| maize starch) | | |
| Lactose DCL-11 (carrier) | 83.40 | 79.00 |
| Avicel PH 101 (carrier, | 45.00 | 45.00 |
| microcrystalline cellulose) | | |
| Ultraamylopectin (disintegrant) | 5.50 | 5.50 |
| Pruv (lubricant, sodium stearyl- | 4.50 | 4.50 |
| fumarate) | | |
| Aerosil 200 (lubricant, colloid | 3.00 | 3.00 |
| silicon dioxide | | |

Tablets are prepared as follows: The weighed amount of Aerosol OT is dissolved in water with the temperature of 70° C. and the resulting solution is cooled to the temperature of 25° C. Finasteride is then suspended in the solution cooled as above. The resulting suspension is milled in a ball mill in order to reach the demanded particle size. Starch 1500, Lactose DCL-11 and Avicel PH 101 are then separately mixed in a mixer and the resulting mixture is transferred into a fluid drier where the finasteride suspension is sprayed onto it. The resulting mixture is then dried at the temperature of 60° C. in order to reach the humidity content not exceeding 3% of the weight. Ultraamylopectin, Pruv and Aerosil 200 are then separately sieved through a sieve having the edge size of 0.3-1.0 mm, and said constituents are mixed in the mixer with the dried mixture containing finasteride as mentioned above. The resulting mixture is then pressed into tablets having the diameter of 7 mm and weighing 150 mg.

### Example 2

The tablets manufactured using the method according to Example 1 are coated with 14% Opadry II—the aqueous pigmented coating dispersion (the hydrophilic coating mixture based on hydroxypropylmethylcellulose and hydroxypropylcellulose) in order to reach the film dry matter of 3.0 mg/tablet.

### Example 3

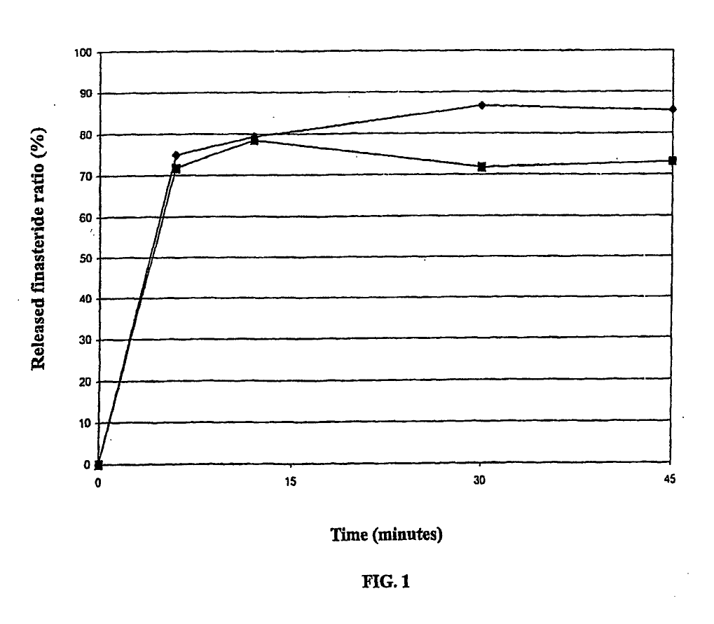

Finasteride release from the tablets manufactured using the method according to Example 1 is determined within the scope of this example as follows. This measurement is performed using the dissolution paddle method in water at the paddle speed of 50 rpm. The amount of finasteride released is determined using HPLC. For the comparison purposes, finasteride release from PROSCAR generic standard is determined under the same conditions. The results obtained are set forth in Table 2 below and their graphical form is expressed in FIG. 1 where values of finasteride released from tablets according to Example 1 are marked with rhombi while the values of finasteride released from PROSCAR generic standard are marked with squares.

TABLE 2

| | Released finasteride ratio (%) | |
|---|---|---|
| Time (minutes) | Example I | PROSCAR |
| 6 | 74.8 | 71.7 |
| 12 | 79.3 | 78.4 |
| 30 | 86.5 | 71.7 |
| 45 | 85.4 | 73.0 |

The progress reached in the solid dosage form prepared using the method according to this invention in comparison with the current prior art is apparent from the obtained results.

1. A method of preparation of an oral solid dosage form with instant release of an active agent containing as the active agent finasteride characterized in that an aqueous suspension containing 5% to 50% by weight of finasteride, based on the total weight of the suspension, and 0.1% to 50% by weight of at least one anion surfactant, based on the weight of finasteride, is milled in order to reach such distribution of particle size of finasteride form that the size of 10% of particles does not exceed 2 μm, the size of 50% of particles does not exceed 7 μm, and the size of 90% of particles does not exceed 17 μm, then the obtained aqueous suspension is sprayed in a fluid bed onto a solid particle hydrophilic carrier having such distribution of particle size that the size of 90% of particles exceeds 40 μm and the size of 10% of particles exceeds 200 μm, and the size of 99% of particles does not exceed 300 μm.

2. The method according to claim 1 characterized in that at least one substance of the following: sodium sulfosuccinate, sodium lauryl sulfate, sodium hexadecylsulfate, sodium hexadecylsulfonate, and sodium dioctysulfosuccinate is used as anion surfactant.

3. The method according to claim 1 characterized in that a hydrophilic sugar, as sucrose, sorbitol, mannitol, glucose and lactose, native or modified starch and cellulose or their mixtures, particularly a mixture of lactose, microcrystalline cellulose and modified maize starch at the weight ratio of 142:86: 11 are used as the solid particle hydrophilic carrier.

4. The method according to claim 3 characterized in that a mixture obtained by the spraying of the aqueous suspension onto the solid particle hydrophilic carrier in the fluid bed is mixed with 2 to 10% by weight, based on the total weight of the obtained mixture, of at least one pharmaceutically acceptable hydrophilic lubricant showing an antistatic effect, such as colloidal silicon dioxide, sodium stearyl fumarate, polyethylene glycol or sodium lauryl sulfate.

5. The method according to claim 4 characterized in that the mixture obtained by the spraing of the aqueous suspension onto the solid particle hydrophilic carrier in the fluid bed is mixed with 1 to 7% by weight, based on the total weight of the obtained mixture, of at least one pharmaceutically acceptable disintegrant, such as ultraamylopectin, cross-linked sodium carboxymethycellulose or cross-linked polyvinylpyrrolidone.

6. The method according to claim 5 characterized in that the mixture obtained by the spraying of the aqueous suspension onto the solid particle hydrophilic carrier in the fluid bed, optionally after being mixed with at least one lubricant and/or with at least one disintegrant, is filled into capsules or sachets or is pressed into tablets.

7. The method according to claim 6 characterized in that the tablets are coated with a water-soluble film or pigmented coating dispersion, particularly the dispersion of the hydrophilic coating mixture based on hydroxypropylmethylcellulose and hydroxypropycellulose wherein the coat weight is 1 to 6% by weight based on the weight of the uncoated tablet.
